(19) United States
(12) Patent Application Publication
(10) Pub. No.: US 2025/0152083 A1
(43) Pub. Date: May 15, 2025

(54) SYSTEM AND METHOD FOR MULTI-MODAL NEUROLOGICAL AND HEALTH ASSESSMENT

(71) Applicant: NeuroVision Imaging, Inc., Sacramento, CA (US)

(72) Inventor: Steven Verdooner, Sacramento, CA (US)

(21) Appl. No.: 18/941,736

(22) Filed: Nov. 8, 2024

Related U.S. Application Data
(60) Provisional application No. 63/597,575, filed on Nov. 9, 2023.

(51) Int. Cl. A61B 5/00; G16H 50/30

(52) U.S. Cl. CPC A61B 5/4076; A61B 5/4803; A61B 5/7264; A61B 5/7275; G16H 50/30

(57) ABSTRACT

A system and method received neurological and lifestyle data from a subject, stores data in a data collection module, adaptively extracts signals associated with neural parameters, dynamically generating neurological risk scores for the subject based using machine learning models, generating recommendations to the subject based on the risk scores, and providing them to the subject.

### CROSS-REFERENCE TO RELATED APPLICATIONS

This application claim priority to U.S. Provisional Application No. 63/597,575 filed Nov. 9, 2023, incorporated by reference herein.

### BACKGROUND OF THE INVENTION

Conventional neurological assessments rely primarily on subjective questionnaires and simple office-based tests, providing limited insights into a patient's cognitive function. There is a need for more quantitative, data-driven approaches that incorporate multiple modalities of data to give a more complete and objective picture of neurological health.

In addition, while neurological conditions are a central indicator of overall well-being, other lifestyle and health factors also play an important role. A holistic understanding of a patient should consider physical health, mental health, diet, exercise, sleep quality, and more.

Accordingly, there is a need for an integrated system that combines neurological assessment with analysis of other health and lifestyle data for a comprehensive and objective evaluation of a patient's cognitive and physical status.

### SUMMARY OF THE INVENTION

The present invention provides a multi-modal health assessment system and method that analyzes data from neurological tests along with speech, blood biomarkers, wearable devices, sleep trackers, exercise trackers, stress monitors, heart rate monitors, diet logs, and more. The system and method generates neurological risk scores and lifestyle scores to objectively quantify a patient's mental and physical wellbeing.

The neurological assessment incorporates speech analysis to detect linguistic biomarkers, blood tests for markers of neurodegeneration, data from wearable sensors, and sleep quality measurements from wearables. The lifestyle assessment includes metrics for physical activity, heart health, stress levels, and diet quality.

A neural assessment module uses machine learning techniques to analyze the diverse data streams and identify any correlations between neurological, physical, and mental health markers. The module computes a set of personalized scores highlighting risks and opportunities to improve the patient's cognitive and overall wellness through targeted recommendations.

### BRIEF DESCRIPTION OF THE DRAWINGS

FIG. 1 is a block diagram of an embodiment of a system architecture which can be used to practice the invention; and

FIG. 2 is a flow chart of method steps of an embodiment which can be used to practice the invention.

### DETAILED DESCRIPTION OF THE PREFERRED EMBODIMENT

The invention provides a system for providing an assessment of a neurological condition of a subject, the system comprising: a plurality of sensors configured to collect biometric data from the subject; at least one processor coupled to the sensors to receive biometric data; a data collection module configured to store the collected biometric data; a neural assessment module configured to: extract a plurality of physiological signals from the stored biometric data; generate one or more neurological risk scores using machine learning models trained on neurological data; and produce personalized health recommendations based on the risk scores.

The plurality of sensors may comprise at least three of: speech sensors; blood sample sensors; wearable motion sensors; sleep monitoring sensors; heart rate sensors; and stress monitoring sensors. The neural assessment module may be further configured to: analyze speech recordings for linguistic biomarkers comprising prosody, lexical complexity, and semantic coherence; and compute a confidence index for the linguistic biomarkers. The neural assessment module may be further configured to: analyze blood samples for biomarkers comprising tau, p-tau, neurofilament-light, glial fibrillary acidic protein (GFAP), amyloid beta, and α-synuclein; and generate risk scores based on detected biomarker levels. The neural assessment module may be further configured to: analyze wearable sensor data comprising heart rate, heart rate variability, blood pressure, sleep patterns, movement analysis, and gait analysis; and identify behavioral patterns indicative of neurological conditions. The machine learning models may comprise: a multi-layer neural network with specific architecture for processing multi-modal data; and trained weights for feature extraction from each sensor type.

The invention provides a method for providing an assessment of a neurological condition of a subject, the method comprising: using a plurality of sensors configured to collect biometric data from the subject; using at least one processor coupled to the sensors to receive biometric data; using a data collection module configured to store the collected biometric data; and using a neural assessment module configured to: extract a plurality of physiological signals from the stored biometric data; generate one or more neurological risk scores using machine learning models trained on neurological data; and produce personalized health recommendations based on the risk scores.

The plurality of sensors may comprise at least three of: speech sensors; blood sample sensors; wearable motion sensors; sleep monitoring sensors; heart rate sensors; and stress monitoring sensors. The neural assessment module may include: analyzing speech recordings for linguistic biomarkers comprising prosody, lexical complexity, and semantic coherence; and computing a confidence index for the linguistic biomarkers. The neural assessment module may include: analyzing blood samples for biomarkers comprising tau, p-tau, neurofilament light, glial fibrillary acidic protein (GFAP), amyloid beta, and a-synuclein; and generating risk scores based on detected biomarker levels. The neural assessment module may include: analyzing wearable sensor data comprising heart rate, heart rate variability, blood pressure, sleep patterns, and gait analysis; and identifying behavioral patterns indicative of neurological conditions. The machine learning models may include: using a multi-layer neural network with specific architecture for processing multi-modal data; and using trained weights for feature extraction from each sensor type.

The invention provides a system for providing an assessment of a neurological condition of a subject, comprising: at least one processor configured to receive and analyze data from a plurality of sensors, wherein the sensors comprise biometric sensors, speech sensors, blood sample sensors, wearable sensors, sleep sensors, exercise sensors, stress sensors, heart rate sensors, and diet sensors; a data collection module configured to store the received data from the sensors; a neural assessment module configured to: adaptively extract a plurality of signals associated with neural parameters of the subject from the data collection module, wherein the signals include speech recordings, biomarker data, and wearable data; dynamically generate one or more neurological risk scores for the subject based on the plurality of signals using machine learning models, wherein the risk scores indicate a probability that the subject suffers from a neurological condition; automatically adjust selection of the neural parameters based on the risk scores; compare a prior clinical assessment to the risk scores to check for false positives or negatives; compute a confidence index for recurring questions posed to the subject and/or a caregiver of the subject; generate predicted improvements in the risk scores based on care recommendations, which are selected to reduce the risk scores; compute trends in the risk scores to determine additional recommendations; reserve care resources based on the risk scores; generate mortality, morbidity, and brain pathology indicators based on the risk scores; generate risk scores for the caregiver based on the subject's risk scores; extract signals from the sensors including biochemical, GPS, respiratory, cardiac, neurological, motion, augmented reality, and blood pressure sensors; analyze the speech recordings for linguistic biomarkers indicative of neurological health; analyze blood biomarkers associated with neurological diseases; analyze wearable data for symptoms and behaviors indicative of neurological health; generate a lifestyle assessment analyzing sleep, exercise, stress, heart health, and diet data; and produce personalized recommendations based on the neurological and lifestyle assessments.

The linguistic biomarkers may comprise features related to prosody, lexical complexity, semantic coherence, and disfluency. The blood biomarkers may comprise tau, p-tau, amyloid beta, α-synuclein, neurofilament light chain, glial fibrillary acidic protein (GFAP), P-53, secreted modular calcium-binding protein (SMOC-1), placental growth factor (PLGF), brain-derived neurotrophic factor (BDNF), and neurogranin. The wearable data may comprise heart rate, sleep staging, gait analysis, tremor, and voice analysis. The machine learning models may comprise neural networks trained on neurological data. The lifestyle assessment may evaluate sleep quality, physical activity, stress levels, heart rate variability, and nutrition intake.

The invention provides a method for providing an assessment of a neurological condition of a subject, comprising: collecting data from biometric, speech, blood, wearable, sleep, exercise, stress, cardiac, and diet sensors; storing the received data from the sensors in a data collection module; adaptively extracting, by a neural assessment module, a plurality of signals associated with neural parameters of the subject from the data collection module, wherein the signals include speech recordings, biomarker data, and wearable data; dynamically generating, by the neural assessment module, one or more neurological risk scores for the subject based on the plurality of signals using machine learning models, wherein the risk scores indicate a probability that the subject suffers from a neurological condition; automatically adjusting selection of the neural parameters based on the risk scores; comparing a prior clinical assessment to the risk scores to check for false positives or negatives; computing a confidence index for recurring questions posed to the subject and/or caregiver; generating predicted improvements in the risk scores based on care recommendations, which are selected to reduce the risk scores; computing trends in the risk scores to determine additional recommendations; reserving care resources based on the risk scores; generating mortality, morbidity, and brain pathology indicators based on the risk scores; generating risk scores for a caregiver based on the subject's risk scores; extracting signals from the sensors including biochemical, GPS, respiratory, cardiac, neurological, motion, augmented reality, and blood pressure sensors; analyzing the speech recordings for linguistic biomarkers indicative of neurological health; analyzing blood biomarkers associated with neurological diseases; analyzing wearable data for symptoms and behaviors indicative of neurological health; generating a lifestyle assessment analyzing sleep, exercise, stress, heart health, and diet data; producing personalized recommendations based on the neurological and lifestyle assessments.

The linguistic biomarkers may comprise prosodic, lexical, semantic, and disfluency features. The blood biomarkers may comprise tau, amyloid beta, a-synuclein, neurofilament light chain, P-53, SMOC-1, PLGF, BDNF, and neurogranin. The wearable data may comprise heart rate, sleep staging, gait, tremor, and voice characteristics. The machine learning models may comprise neural networks trained on neurological data. The lifestyle assessment may evaluate sleep quality, activity, stress, heart rate variability, blood pressure, and nutrition.

A preferred embodiment will be described as an exemplary way to practice the Invention, but the invention is not limited to this embodiment.

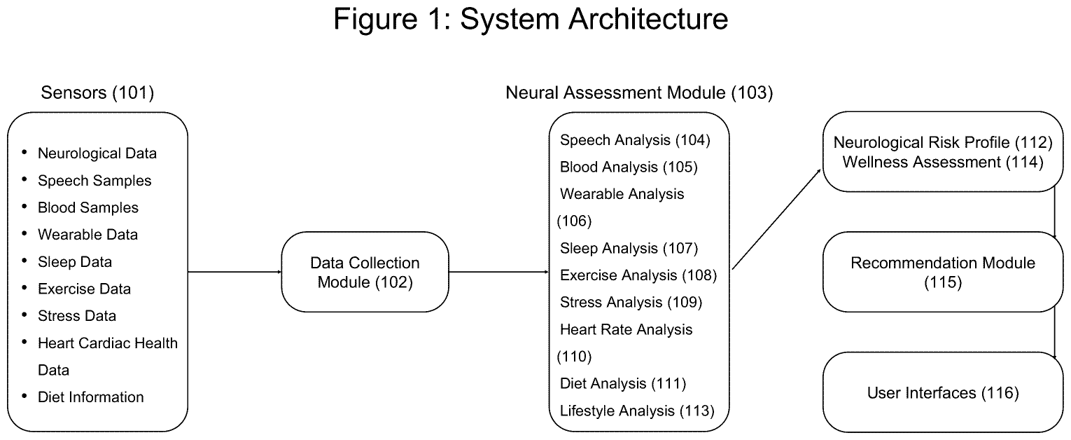

FIG. 1 illustrates an example system architecture, with multiple sensors measuring neurological, physical, and mental health data from a patient, which feeds into a computer system having a processor that analyzes the data, generates health profiles and recommendations, and displays them to the patient and providers. Sensors 101 collect neurological data, speech samples, blood samples, wearable data, sleep data, exercise data, stress data, heart health data, and diet information. Examples of different sensors used to collect neurological, physical, and mental health data, include wearable EEG/ECG, finger-worn pulse oximeter, wrist actigraphy/heart rate monitor, chest patch EMG, headband sleep tracker, mobile EEG, smart footwear, and ingestible biochemical sensor, attached to the head, chest, and wrist for example. A data collection module 102 stores the multimodal data.

A neural assessment module 103 applies machine learning algorithms to analyze the data. Neural network architecture and training within the machine learning models can be used for multi-modal data analysis and health profile generation. Speech analysis 104 extracts linguistic biomarkers indicating neurological function. Blood analysis 105 measures neurodegenerative proteins and metabolites. Wearable analysis 106 extracts symptoms and behaviors. Sleep analysis 107 characterizes sleep quality. Exercise analysis 108 quantifies activity levels. Stress analysis 109 computes stress indicators. Heart rate analysis 110 calculates heart rate variability metrics. Diet analysis 111 evaluates nutrition intake.

The neural assessment module 103 combines insights from the data analyses to generate a neurological risk profile 112 for the patient. Lifestyle analysis 113 evaluates physical activity, sleep, stress, and diet metrics to produce an overall wellness assessment 114.

A recommendation module 115 uses the neurological risk profile 112 and wellness assessment 114 to generate personalized recommendations to improve the patient's cognitive health and overall wellbeing through targeted interventions. The recommendations are provided to the patient and care providers via user interfaces 116. A personalized recommendation dashboard can be displayed to a user, using a screen displaying graphs or profiles with sections for cognitive scores, physical scores, prioritized recommendations, and tips to improve risk areas.

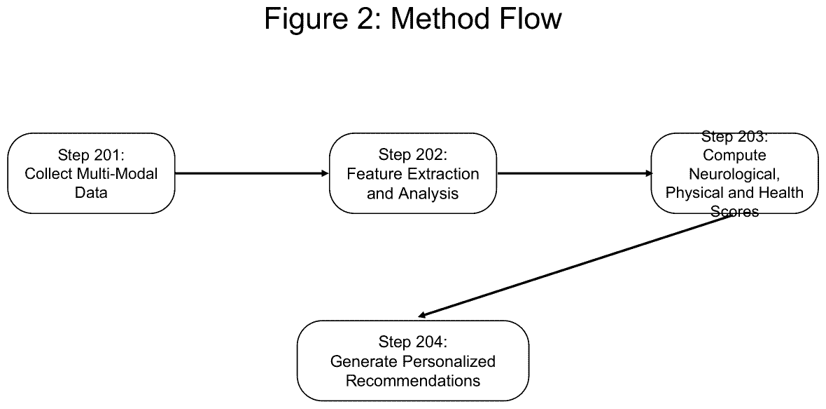

FIG. 2 shows an example method, with a high-level process flow with steps to: (1) collect multi-modal health data, (2) analyze data to extract biomarkers and metrics, (3) compute neurological and lifestyle health profiles, and (4) generate personalized recommendations. Step 201 collects multi-modal data from sensors, speech recordings, blood tests, etc. Step 202 performs feature extraction and analysis on the data. Step 203 computes neurological, physical, and mental health scores. Step 204 generates personalized recommendations to lower risks and enhance wellness. A personalized recommendation dashboard can be displayed to a user, with sections for cognitive scores, physical scores, prioritized recommendations, and tips to improve risk areas. The information can be sent to both a patient and a provider, possibly via mobile devices or computer screens.

By providing both neurological analysis and lifestyle evaluation, the system delivers a holistic and objective assessment of mental and physical wellbeing. The data fusion and machine learning techniques enable objective personalized and quantitative insights unmatched by conventional subjective evaluations. This allows customized interventions to improve cognitive function and overall health.

More details on the data collection and analysis will be provided below.

### Data Collection Protocol

### Speech Analysis

1. Record minimum 3-minute speech samples using standardized tasks:
   Picture description task (1 minute)
   Category fluency test (1 minute)
   Free speech narrative (1 minute)
2. Extract acoustic features:
   Fundamental frequency (F0) statistics: mean, variance, range
   Speaking rate: syllables per second
   Pause metrics: number, duration, distribution
   Voice quality measures: jitter, shimmer, harmonics-to-noise ratio

### Blood Biomarker Analysis

1. Collect 10 mL blood sample using standard venipuncture
2. Process within 2 hours of collection:
   Centrifuge at 2000 g for 15 minutes at 4° C.
   Separate plasma and store at −80° C.
3. Measure using standardized assays:
   Tau protein: Enzyme-Linked ImmunoSorbent Assay (ELISA) method, threshold >242 μg/mL
   Amyloid beta: Mass spectrometry, threshold >42 μg/mL
   α-synuclein: Immunoassay, threshold >1.5 ng/ml

### Wearable Data Collection

1. Minimum 72-hour continuous monitoring period
2. Required metrics:
   a. Heart rate: Sample every 1 second
   b. Heart rate variability: Sample every 1 second
   c. Blood pressure: Sample every hour
   d. Activity: Steps, movement intensity every 1 minute
   e. Sleep: Continuous monitoring of sleep stages
   f. Location: GPS coordinates every 5 minutes

### Signal Processing Pipeline

### Speech Processing

1. Pre-processing:
   Noise reduction using spectral subtraction
   Silence removal with −20 dB threshold
   Segmentation into 20 ms frames
2. Feature extraction:
   Mel-Frequency Cepstral Coefficients (MFCC) coefficients (13 dimensions)
   Prosodic features (pitch, energy, duration)
   Linguistic features (word frequency, complexity)

### Wearable Data Processing

1. Signal cleaning:
   Median filtering for outlier removal
   Interpolation of missing data (<10% gaps)
   Moving average smoothing (5-point window)
2. Feature extraction:
   Heart rate variability metrics
   Blood pressure variability metrics
   Activity bout classification
   Sleep stage transitions
3. Risk Score Computation

### Neural Network Architecture

1. Input layer:
   Speech features (64 nodes)
   Blood biomarkers (32 nodes)
   Wearable features (128 nodes)
2. Hidden layers:
   Layer 1:256 nodes, Rectified Linear Unit (ReLU) activation
   Layer 2:128 nodes, ReLU activation
   Layer 3:64 nodes, ReLU activation
3. Output layer:
   o 5 nodes (risk scores), sigmoid activation

### Risk Score Calculation

1. Individual domain scores:
   Speech risk=Σ(wi×si) where wi-feature weights, si=speech features
   Blood risk=Σ(bi×mi) where bi=biomarker weights, mi=marker levels
   Wearable risk=Σ(vi×di) where vi=vital weights, di=device features
2. Combined risk score:
   a. Final risk=0.4×Speech+0.3×Blood+0.3×Wearable
   b. Normalize to 0-100 scale

### Recommendation Generation

1. Risk threshold triggers:
   Low risk: <30
   Moderate risk: 30-70
High risk: >70
2. Recommendation rules:
   High risk→Weekly specialist monitoring
Moderate risk→Monthly check-ins
Low risk→Quarterly assessments

### Quality Control

1. Data validation:
   Minimum 80% data completeness required
   Signal quality metrics must exceed 0.7
   Automated artifact detection
2. Confidence scoring:
   Bootstrap sampling (1000 iterations)
   Confidence interval calculation
   Minimum 85% confidence required for risk score

### Machine Learning Module Implementation

The system implements multiple machine learning modules within both the neural assessment module 103 and recommendation module 115. An exemplary implementation of a machine learning module for neurological risk assessment is detailed below, along with the steps that one skilled in the art would follow to develop and deploy such modules.
1. Data Preparation Phase
   Collect training dataset comprising minimum 10,000 patient records
   Each record includes:
      Speech features (MFCC coefficients, prosodic measures)
      Blood biomarker levels (tau, p-tau, amyloid beta, etc.)
      Wearable sensor data (heart rate, activity, sleep patterns)
      Clinical diagnosis labels (validated by medical professionals)
   Perform data cleaning:
      Remove records with >20% missing values
      Normalize numerical features to 0-1 range
      Encode categorical variables using one-hot encoding
   Split dataset: 70% training, 15% validation, 15% testing
2. Model Architecture
   Input Layer:
      Separate branches for each data modality
      Speech branch: 1D convolutional layers (kernel size=3, stride=1)
      Blood branch: Fully connected layers with batch normalization
      Wearable branch: LSTM layers for temporal data processing
   Feature Fusion Layer:
      Concatenate outputs from all branches
      Apply attention mechanism to weight different modalities
   Classification Layers:
      Three fully connected layers (256, 128, 64 nodes)
      Dropout layers (rate=0.3) between fully connected layers
      Output layer: Sigmoid activation for risk score prediction
3. Training Protocol
   Initialize weights using Xavier/Glorot initialization
   Optimize using Adam optimizer:
      Learning rate: 0.001
      Beta1: 0.9
      Beta2: 0.999
   Loss function: Binary cross-entropy with L2 regularization
   Training schedule:
      Batch size: 32
      Maximum epochs: 100
      Early stopping patience: 10 epochs
      Learning rate reduction on plateau (factor-0.5)
4. Validation and Testing
   Performance metrics:
      Area Under ROC Curve (AUC)
      Sensitivity and specificity
      F1 score
      Confidence intervals using bootstrap sampling
   Minimum acceptable performance:
      AUC >0.85
      Sensitivity >0.80
      Specificity >0.85
   Cross-validation using 5-fold strategy
5. Model Deployment
   Export trained model using ONNX format
   Implement inference pipeline:
      Real-time data preprocessing
      Feature extraction matching training pipeline
      Model inference with batch processing
      Risk score calibration using Platt scaling
   Quality control:
      Monitor prediction distribution drift
      Track confidence scores
      Log all predictions for audit purposes
6. Continuous Learning
   Collect new labeled data monthly
   Retrain model quarterly using:
      Combined historical and new data
      Transfer learning from previous model
   Validate performance improvements:
      Compare metrics with previous version
   Conduct A/B testing on subset of cases
   Require minimum 2% improvement for deployment

The implementation follows industry best practices for medical AI systems, ensuring reproducibility and clinical validity. One skilled in the art would follow these steps to implement the machine learning modules, adjusting hyperparameters and architecture based on available data and specific use cases.

The recommendation module 115 uses a similar implementation approach but with modified architecture focusing on mapping risk scores to intervention recommendations using reinforcement learning techniques.

Although a preferred embodiment has been described, variations will occur to those skilled in the art, and the scope of the invention is not limited by the embodiment but only by the claims.

1. A system for providing an assessment of a neurological condition of a subject, the system comprising:
a plurality of sensors configured to collect biometric data from the subject;
at least one processor coupled to the sensors to receive biometric data;
a data collection module configured to store the collected biometric data;
a neural assessment module configured to:
extract a plurality of physiological signals from the stored biometric data;
generate one or more neurological risk scores using machine learning models trained on neurological data; and
produce personalized health recommendations based on the risk scores.

2. The system of claim 1, wherein the plurality of sensors comprises at least three of:
speech sensors;
blood sample sensors;
wearable motion sensors;
sleep monitoring sensors;
heart rate sensors;
stress monitoring sensors.

3. The system of claim 1, wherein the neural assessment module is further configured to:
analyze speech recordings for linguistic biomarkers comprising prosody, lexical complexity, and semantic coherence; and
compute a confidence index for the linguistic biomarkers.

4. The system of claim 1, wherein the neural assessment module is further configured to:
analyze blood samples for biomarkers comprising tau, p-tau, neurofilament-light, glial fibrillary acidic protein (GFAP), amyloid beta, and a-synuclein; and
generate risk scores based on detected biomarker levels.

5. The system of claim 1, wherein the neural assessment module is further configured to:
analyze wearable sensor data comprising heart rate, heart rate variability, blood pressure, sleep patterns, movement analysis, and gait analysis; and
identify behavioral patterns indicative of neurological conditions.

6. The method of claim 1, wherein the machine learning models comprise:
a multi-layer neural network with specific architecture for processing multi-modal data; and
trained weights for feature extraction from each sensor type.

7. A method for providing an assessment of a neurological condition of a subject, the method comprising:
using a plurality of sensors configured to collect biometric data from the subject;
using at least one processor coupled to the sensors to receive biometric data;
using a data collection module configured to store the collected biometric data; and
using a neural assessment module configured to:
extract a plurality of physiological signals from the stored biometric data;
generate one or more neurological risk scores using machine learning models trained on neurological data; and
produce personalized health recommendations based on the risk scores.

8. The method of claim 7, wherein the plurality of sensors comprises at least three of:
speech sensors;
blood sample sensors;
wearable motion sensors;
sleep monitoring sensors;
heart rate sensors;
stress monitoring sensors.

9. The method of claim 7, wherein using a neural assessment module includes:
analyzing speech recordings for linguistic biomarkers comprising prosody, lexical complexity, and semantic coherence; and
computing a confidence index for the linguistic biomarkers.

10. The method of claim 7, wherein using a neural assessment module includes:
analyzing blood samples for biomarkers comprising tau, p-tau, neurofilament light, glial fibrillary acidic protein (GFAP), amyloid beta, and a-synuclein; and
generating risk scores based on detected biomarker levels.

11. The method of claim 7, wherein using a neural assessment module includes:
analyzing wearable sensor data comprising heart rate, heart rate variability, blood pressure, sleep patterns, and gait analysis; and
identifying behavioral patterns indicative of neurological conditions.

12. The method of claim 7, wherein using machine learning models includes:
using a multi-layer neural network with specific architecture for processing multi-modal data; and
using trained weights for feature extraction from each sensor type.

13. A system for providing an assessment of a neurological condition of a subject, the system comprising:
at least one processor configured to receive and analyze data from a plurality of sensors, wherein the sensors comprise biometric sensors, speech sensors, blood sample sensors, wearable sensors, sleep sensors, exercise sensors, stress sensors, heart rate sensors, and diet sensors;
a data collection module configured to store the received data from the sensors;
a neural assessment module configured to:
adaptively extract a plurality of signals associated with neural parameters of the subject from the data collection module, wherein the signals include speech recordings, biomarker data, and wearable data;
dynamically generate one or more neurological risk scores for the subject based on the plurality of signals using machine learning models, wherein the risk scores indicate a probability that the subject suffers from a neurological condition;
automatically adjust selection of the neural parameters based on the risk scores;
compare a prior clinical assessment to the risk scores to check for false positives or negatives;
compute a confidence index for recurring questions posed to the subject and/or a caregiver of the subject;
generate predicted improvements in the risk scores based on care recommendations, which are selected to reduce the risk scores;
compute trends in the risk scores to determine additional recommendations;
reserve care resources based on the risk scores;
generate mortality, morbidity, and brain pathology indicators based on the risk scores;
generate risk scores for the caregiver based on the subject's risk scores;
extract signals from the sensors including biochemical, GPS, respiratory, cardiac, neurological, motion, augmented reality, and blood pressure sensors;
analyze the speech recordings for linguistic biomarkers indicative of neurological health;
analyze blood biomarkers associated with neurological diseases;
analyze wearable data for symptoms and behaviors indicative of neurological health;
generate a lifestyle assessment analyzing sleep, exercise, stress, heart health, and diet data; and
produce personalized recommendations based on the neurological and lifestyle assessments.

14. The system of claim 13, wherein the linguistic biomarkers comprise features related to prosody, lexical complexity, semantic coherence, and disfluency.

15. The system of claim 13, wherein the blood biomarkers comprise tau, p-tau, amyloid beta, α-synuclein, neurofilament light chain, glial fibrillary acidic protein (GFAP), P-53, secreted modular calcium-binding protein (SMOC-1), placental growth factor (PLGF), brain-derived neurotrophic factor (BDNF), and neurogranin.

16. The system of claim 13, wherein the wearable data comprises heart rate, sleep staging, gait analysis, tremor, and voice analysis.

17. The system of claim 13, wherein the machine learning models comprise neural networks trained on neurological data.

18. The system of claim 13, wherein the lifestyle assessment evaluates sleep quality, physical activity, stress levels, heart rate variability, and nutrition intake.

19. A method for providing an assessment of a neurological condition of a subject, comprising:
collecting data from biometric, speech, blood, wearable, sleep, exercise, stress, cardiac, and diet sensors;
storing the received data from the sensors in a data collection module;
adaptively extracting, by a neural assessment module, a plurality of signals associated with neural parameters of the subject from the data collection module, wherein the signals include speech recordings, biomarker data, and wearable data;
dynamically generating, by the neural assessment module, one or more neurological risk scores for the subject based on the plurality of signals using machine learning models, wherein the risk scores indicate a probability that the subject suffers from a neurological condition;
automatically adjusting selection of the neural parameters based on the risk scores;
comparing a prior clinical assessment to the risk scores to check for false positives or negatives;
computing a confidence index for recurring questions posed to the subject and/or caregiver;
generating predicted improvements in the risk scores based on care recommendations, which are selected to reduce the risk scores;
computing trends in the risk scores to determine additional recommendations;
reserving care resources based on the risk scores;
generating mortality, morbidity, and brain pathology indicators based on the risk scores;
generating risk scores for a caregiver based on the subject's risk scores;
extracting signals from the sensors including biochemical, GPS, respiratory, cardiac, neurological, motion, augmented reality, and blood pressure sensors;
analyzing the speech recordings for linguistic biomarkers indicative of neurological health;
analyzing blood biomarkers associated with neurological diseases;
analyzing wearable data for symptoms and behaviors indicative of neurological health;
generating a lifestyle assessment analyzing sleep, exercise, stress, heart health, and diet data;
producing personalized recommendations based on the neurological and lifestyle assessments.

20. The method of claim 19, wherein the linguistic biomarkers comprise prosodic, lexical, semantic, and disfluency features.

21. The method of claim 19, wherein the blood biomarkers comprise tau, amyloid beta, α-synuclein, neurofilament light chain, P-53, SMOC-1, PLGF, BDNF, and neurogranin.

22. The method of claim 19, wherein the wearable data comprises heart rate, sleep staging, gait, tremor, and voice characteristics.

23. The method of claim 19, wherein the machine learning models comprise neural networks trained on neurological data.

24. The method of claim 19, wherein the lifestyle assessment evaluates sleep quality, activity, stress, heart rate variability, blood pressure, and nutrition.
